(19) United States
(12) Patent Application Publication
(10) Pub. No.: US 2024/0150723 A1
(43) Pub. Date: May 9, 2024

(54) REDUCING HOST CELL IMPURITIES DURING RECOMBINANT PROTEIN PRODUCTION

(71) Applicant: Biogen MA Inc., Cambridge, MA (US)

(72) Inventors: Taylor Forte, Cambridge, MA (US); Christoper Kwiatkowski, Cambridge, MA (US); Greg Evangelist, Cary, NC (US); Taylor Roland, Morrisville, NC (US); Krishnakumar Malu, Cambridge, MA (US)

(73) Assignee: Biogen MA Inc., Cambridge, MA (US)

(21) Appl. No.: 18/283,614

(22) PCT Filed: Mar. 22, 2022
(86) PCT No.: PCT/US22/21313

§ 371 Date: Sep. 22, 2023

Related U.S. Application Data
(60) Provisional application No. 63/164,988, filed on Mar. 23, 2021.

(51) Int. Cl. C12N 5/071

(52) U.S. Cl. CPC C12N 5/0682; C12N 2500/24; C12N 2500/30; C12N 2523/00

(57) ABSTRACT

In some instances, the application provides methods of culturing host cells comprising a gene encoding a recombinant N protein in a cell culture medium, wherein the cell culture medium comprises: (i) iron at a concentration of less than 1200 μM; and (ii) citrate at a concentration of less than 2400 μM. In some instances, the methods comprise culturing host cells expressing a recombinant protein in a cell culture medium at a first temperature; and decreasing the first temperature to a second temperature. In some instances, the methods comprise purifying the recombinant protein by a carbon depth filtration.

### RELATED APPLICATIONS

This application claims priority under 35 U.S.C. § 119(e) to U.S. Provisional Patent Application No. 63/164,988, filed Mar. 23, 2021, the entire contents of which are incorporated herein by reference.

### BACKGROUND

Host cell proteins (HCPs) are process-related impurities associated with the production of recombinant proteins. These impurities can negatively impact the stability, safety, and efficacy of the therapeutic protein and can be challenging to remove.

### SUMMARY

The present application, at least in part, provides methods for reducing host cell proteins (e.g., HSP90 and/or perilipin) during recombinant protein production. In some instances, methods and compositions have little or no material impact on cell viability and/or titer of the recombinant protein. In some instances, the methods comprise modulating one or more cell culture conditions (e.g., temperature, iron concentration, citrate concentration) in an upstream production process. Accordingly, in some instances, the methods comprise culturing host cells comprising a gene encoding a recombinant protein in a cell culture medium. In some instances, the cell culture medium comprises iron at a concentration of less than 2400 μM. In some instances, the cell culture medium comprises citrate at a concentration of less than 8500 μM. In some instances, the cell culture medium comprises iron at a concentration of less than 1200 μM. In some instances, the cell culture medium comprises citrate at a concentration of less than 2400 μM. In some instances, the methods comprise culturing host cells expressing a recombinant protein in a cell culture medium at a first temperature; and decreasing the first temperature to a second temperature. In some instances, the second temperature is lower than 31° C. In some instances, the methods further comprise modulating one or more conditions in a downstream production process (e.g., purification of a recombinant protein). In some instances, the methods further comprise purifying the recombinant protein by a carbon depth filtration.

In some instances, the present application provides a method comprising: culturing host cells comprising a gene encoding a recombinant protein in a cell culture medium, wherein the cell culture medium comprises: (i) iron at a concentration of less than 1200 μM; and (ii) citrate at a concentration of less than 2400 μM.

In some instances, the cell culture medium comprises iron at a concentration of between about 700 μM and about 1100 μM. In some instances, the cell culture medium comprises iron at a concentration of between about 1000 μM and about 1100 μM. In some instances, the cell culture medium comprises citrate at a concentration of between about 1400 μM and about 2200 μM. In some instances, the cell culture medium comprises citrate at a concentration of between about 2100 μM and about 2200 μM.

In some instances, the present application provides a method comprising: culturing host cells expressing a recombinant protein in a cell culture medium at a first temperature; and decreasing the first temperature to a second temperature, wherein the second temperature is lower than 31° C.

In some instances, the host cells are cultured at the first temperature for at least 1 day prior to decreasing to the second temperature. In some instances, the host cells are cultured at the first temperature for approximately 2 days prior to decreasing to the second temperature. In some instances, the decreasing of the first temperature occurs on day 3 of a production bioreactor process. In some instances, the first temperature is between about 34° C. and about 36° C. In some instances, the first temperature is 35° C. In some instances, the second temperature is between about 28° C. and about 30° C. In some instances, the second temperature is 29° C.

In some instances, the present application provides a method comprising: (i) culturing host cells expressing a recombinant protein in a cell culture medium at a first temperature, wherein the cell culture medium comprises iron at a concentration of less than 1200 M and citrate at a concentration of less than 2400 μM; and (ii) decreasing the first temperature to a second temperature, wherein the second temperature is lower than 31° C.

In some instances, the cell culture medium comprises iron at a concentration of between about 700 μM and about 1100 μM. In some instances, the cell culture medium comprises iron at a concentration of between about 1000 μM and about 1100 μM. In some instances, the cell culture medium comprises citrate at a concentration of between about 1400 μM and about 2200 μM. In some instances, the cell culture medium comprises citrate at a concentration of between about 2100 μM and about 2200 μM. In some instances, the cell culture medium is a chemically-defined cell culture medium. In some instances, the cell culture medium is a serum-free cell culture medium. In some instances, the cell culture medium is a chemically-defined, serum-free cell culture medium. In some instances, the cell culture medium does not comprise transferrin.

In some instances, the host cells are cultured at the first temperature for at least 1 day prior to decreasing to the second temperature. In some instances, the host cells are cultured at the first temperature for approximately 2 days prior to decreasing to the second temperature. In some instances, the decreasing of the first temperature occurs on day 3 of a production bioreactor process. In some instances, the first temperature is between about 34° C. and about 36° C. In some instances, the first temperature is 35° C. In some instances, the second temperature is between about 28° C. and about 30° C. In some instances, the second temperature is 29° C.

In some instances, a method of the application further comprises purifying the recombinant protein by a carbon depth filtration.

In some instances, the host cells are mammalian cells. In some instances, the host cells are mammalian cells selected from the group consisting of: Chinese hamster ovary (CHO) cells, human embryonic kidney (HEK) cells, verda reno (VERO) cells, nonsecreting null (NSO) cells, human embryonic retinal (PER.C6) cells, Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells.

In some instances, the recombinant protein is an antibody, an immunoadhesin, a Transforming Growth Factor (TGF) beta superfamily signaling molecule, a blood clotting factor, a Fc fusion protein, combinations thereof, or fragments thereof. In some instances, the recombinant protein comprises an ATP binding site. In some instances, the recombinant protein binds a target molecule comprising an ATP binding site. In some instances, the recombinant protein is an anti-α-synuclein antibody.

In some instances, the method reduces host cell protein levels by at least 30%. In some instances, the host cell protein is heat shock protein 90 (HSP90) and/or perilipin.

### BRIEF DESCRIPTION OF THE DRAWINGS

FIGS. 1A-1C show the HCP level in drug substance using CHO host cell A and CHO host cell B. FIG. 1A shows the total HCP species in drug substance produced by either CHO host cell A or CHO host cell B. FIG. 1B shows perilipin (HCP X) in drug substance produced by either CHO host cell A or CHO host cell B. FIG. 1C shows HSP90 in drug substance produced by either CHO host cell A or CHO host cell B.

FIGS. 2A-2E show the mechanism of the cellular signaling perilipin may be involved in with respect to reactive oxygen species (ROS) generation and iron/citrate concentration in the cell culture medium. FIG. 2A shows that CHO cell host B produced higher levels of perilipin compared to CHO cell host A. FIG. 2B shows that there were higher levels of fatty acid β-oxidation I in CHO host cell B compared to CHO host cell A. FIG. 2C shows that free iron in cell culture medium may be related to ROS generation. FIG. 2D shows that CHO host cell B exhibited lower iron update compared to CHO host cell A. FIG. 2E shows that CHO host cell B exhibited higher iron export compared to CHO host cell A.

FIGS. 3A-3D show the impact of low temperature, low iron/citrate or low temperature/iron/citrate on free iron level in the cell medium, viable cell density (VCD), cell viability and antibody titer. FIG. 3A shows the impact of low temperature, low iron/citrate or low temperature/iron/citrate on free iron level in the cell medium. FIG. 3B shows the impact of low temperature, low iron/citrate or low temperature/iron/citrate on viable cell density (VCD).

FIG. 3C shows the impact of low temperature, low iron/citrate or low temperature/iron/citrate on cell viability. FIG. 3D shows the impact of low temperature, low iron/citrate or low temperature/iron/citrate on antibody titer.

FIGS. 4A-4B show HSP90 and Perilipin levels in ProA eluate after CHO host cell B was cultured in low temperature or low temperature/iron/citrate conditions. FIG. 4A shows HSP90 levels in ProA eluate after CHO host cell B was cultured in low temperature or low temperature/iron/citrate conditions. FIG. 4B shows Perilipin levels in ProA eluate after CHO host cell B was cultured in low temperature or low temperature/iron/citrate conditions.

FIGS. 5A-5F show the effects of downstream purification process on clearance of perilipin (HCP 1) and HSP90 (HCP 2). FIG. 5A shows that protein A column wash was effective in reducing (e.g., removing) perilipin under high arginine concentration at low pH with a yield loss, and was not effective in reducing/removing HSP90. FIG. 5B shows that anion exchange chromatography (AEX) was effective in reducing (e.g., removing) perilipin but not HSP90. FIG. 5C shows that hydrophobic interaction chromatography (HIC) was not effective in removing HSP90. FIG. 5D shows the effect of carbon depth filtration conditions (loading mass, pH and salt concentration) on clearance of perilipin. FIG. 5E shows the effect of carbon depth filtration conditions (loading mass, pH and salt concentration) on clearance of perilipin. FIG. 5F shows carbon and synthetic depth filters are effective in removing perilipin. The carbon depth filter cleared more HSP90 compared to the synthetic filter.

FIGS. 6A-6C show that a combination of upstream cell culture modulation and downstream purification process was effective in removing perilipin (HCP-1) and HSP90 (HCP-2) from the drug substance. FIG. 6A shows the flow of a designed pilot run. FIGS. 6B-6C show the levels of HCP-1 and HCP-2 in control conditions run (pilot 1) and after the process was modified for HCP reduction in a modified run (pilot 2). During process modification, the cell culture medium was modified by reducing levels of iron and citrate (ferric citrate), the culture temperature was reduced as described in Example 2, and carbon depth filters were used in purification. As a result of these process levers used in combination, the amount of HSP90 and perilipin were reduced in the overall process (e.g., HCCF, all in-process intermediates, and the drug substance).

### DETAILED DESCRIPTION

Host cell proteins (HCPs) are process-related impurities associated with the production of recombinant proteins. These impurities can negatively impact the stability, safety, and efficacy of the therapeutic protein and can be challenging to remove. The present application, at least in part, provides methods for reducing host cell proteins (e.g., HSP90 and/or perilipin) during recombinant protein production. In some instances, methods and compositions have little or no material impact on cell viability and/or titer of the recombinant protein. In some instances, the methods comprise modulating one or more cell culture conditions (e.g., temperature, iron concentration, citrate concentration) in an upstream production process. In some instances, the methods comprise culturing host cells comprising a gene encoding a recombinant protein in a cell culture medium, wherein the cell culture medium comprises a reduced concentration of iron (e.g., less than 1200 μM) and/or citrate (e.g., less than 2400 μM). In some instances, the methods comprise culturing host cells expressing a recombinant protein in a cell culture medium at a first temperature; and decreasing the first temperature to a second temperature, wherein the second temperature is lower than 31° C. In some instances, the methods comprise modulating one or more conditions in a downstream purification process of the recombinant protein. In some instances, the methods comprise purifying the recombinant protein by a carbon depth filtration. In some instances, the methods provided herein are useful for making recombinant proteins with reduced levels of contaminating HCP(s). In some instances, the reduction of HCP(s) can be useful to produce a cell lysate with lower levels of HCPs, and in turn can be helpful to purify recombinant proteins with lower levels of contaminating HCP(s).

### I. Modulation of Upstream Cell Culture Conditions

In some instances, the present application describes a method of culturing host cells comprising a gene encoding a recombinant protein in a cell culture medium at a temperature lower than a control cell culture, and the cell culture medium comprises lower iron and/or citrate concentrations compared to conditions of a control cell culture. In some instances, the low temperature and/or low iron/citrate cell culture medium described herein reduces certain host cell proteins (e.g., heat shock protein 90 (HSP90), and/or perilipin) as compared to a control cell culture. For example, in some instances, methods of the application provide a reduction in host cell proteins as compared to a control cell culture performed in a culture medium comprising a higher concentration of iron (e.g., higher than 1200 μM) and/or a higher concentration of citrate (e.g., higher than 2400 μM). In some instances, methods of the application provide a reduction in host cell proteins as compared to a control cell culture performed by decreasing the first temperature to a second temperature that is higher than 31° C. In some instances, methods of the application provide a reduction in host cell proteins as compared to a control purification process performed by purifying a recombinant protein using means other than a carbon depth filtration (e.g., using a synthetic depth filter).

In some instances, the method comprises selecting a host cell for production of a recombinant protein. Various methods of preparing host cells for production of proteins or polypeptides by batch and fed-batch culture are well known in the art. A nucleic acid sufficient to achieve expression (typically a vector containing the gene encoding the polypeptide or protein of interest and any operably linked genetic control elements) can be introduced into the host cell line by any number of well-known techniques. Typically, cells are screened to determine which of the host cells have actually taken up the vector and express the polypeptide or protein of interest. Traditional methods of detecting a particular polypeptide or protein of interest expressed by host cells include but are not limited to immunohistochemistry, immunoprecipitation, flow cytometry, immunofluorescence microscopy, SDS-PAGE, Western blots, enzyme-linked immunosorbent assay (ELISA), high performance liquid chromatography (HPLC) techniques, biological activity assays and affinity chromatography. One of ordinary skill in the art will be aware of other appropriate techniques for detecting expressed polypeptides or proteins. If multiple host cells express the polypeptide or protein of interest, some or all of the listed techniques can be used to determine which of the cells expresses that polypeptide or protein at the highest levels. In some instances, the host cells are mammalian cells. Examples of mammalian cells suitable as host cells include, without limitation, Chinese hamster ovary (CHO) cells, human embryonic kidney (HEK) cells, verda reno (VERO) cells, nonsecreting null (NSO) cells, human embryonic retinal (PER.C6) cells, Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells.

Once a cell that expresses the polypeptide or protein of interest has been identified, the cell is propagated in culture by any of the variety of methods well-known to one of ordinary skill in the art. The cell expressing the polypeptide of interest is typically propagated by growing it at a temperature and in a medium that is conducive to the survival, growth and viability of the cell, and the production of the recombinant protein. The initial culture volume can be of any size, but is often smaller than the culture volume of the production bioreactor used in the final production of the polypeptide or protein of interest, and frequently cells are passaged several times in bioreactors of increasing volume prior to seeding the production bioreactor. The cell culture can be agitated or shaken to increase oxygenation of the medium and dispersion of nutrients to the cells. Alternatively or additionally, special sparging devices that are well known in the art can be used to increase and control oxygenation of the culture. In accordance with the present application, one of ordinary skill in the art will understand that it can be beneficial to control or regulate certain internal conditions of the bioreactor, including but not limited to pH, temperature, oxygenation, etc.

The term “bioreactor” as used herein, in some instances, refers to any vessel used for the growth of a mammalian cell culture. The bioreactor can be of any size so long as it is useful for the culturing of mammalian cells. Typically, the bioreactor will be at least 1 liter and can be 10, 50, 100, 250, 500, 1,000, 2,000, 2,500, 3,000, 5,000, 8,000, 10,000, 12,000, 15,000, 20,000, 30,000 liters or more, or any volume in between. For example, a bioreactor will be 10 to 5,000 liters, 10 to 10,000 liters, 10 to 15,000 liters, 10 to 20,000 liters, 10 to 30,000 liters, 50 to 5,000 liters, 50 to 10,000 liters, 50 to 15,000 liters, 50 to 20,000 liters, 50 to 30,000 liters, 1,000 to 5,000 liters, or 1,000 to 3,000 liters. The internal conditions of the bioreactor, including, but not limited to pH and temperature, are typically controlled during the culturing period. The bioreactor can be composed of any material that is suitable for holding mammalian cell cultures suspended in media under the culture conditions of the present application, including glass, plastic or metal. The term “production bioreactor” as used herein refers to the final bioreactor used in the production of a polypeptide or protein of interest. The volume of the large-scale cell culture production bioreactor is typically at least 500 liters and can be 1,000, 2,000, 2,500, 5,000, 8,000, 10,000, 12,000, 15,000 liters or more, or any volume in between. For example, the large scale cell culture reactor will be between about 500 liters and about 20,000 liters, about 500 liters and about 10,000 liters, about 500 liters and about 5,000 liters, about 1,000 liters and about 30,000 liters, about 2,000 liters and about 30,000 liters, about 3,000 liters and about 30,000 liters, about 5,000 liters and about 30,000 liters, or about 10,000 liters and about 30,000 liters, or a large scale cell culture reactor will be at least about 500 liters, at least about 1,000 liters, at least about 2,000 liters, at least about 3,000 liters, at least about 5,000 liters, at least about 10,000 liters, at least about 15,000 liters, or at least about 20,000 liters. One of ordinary skill in the art will be aware of and will be able to choose suitable bioreactors for use in practicing the methods of the present disclosure.

The cell density useful in the methods of the present application can be chosen by one of ordinary skill in the art. In accordance with the present application, the cell density can be as low as a single cell per culture volume. In some instances, starting cell densities (seed density) can range from about 2×10² viable cells per mL to about 2×10³, 2×10⁴, 2×10⁵, 2×10⁶, 5×10⁶ or 10×10⁶ viable cells per mL and higher.

The terms “culture” and “cell culture” as used herein, in some instances, refer to a cell population, either surface-attached or in suspension that is maintained or grown in a medium under conditions suitable to survival and/or growth of the cell population. As will be clear to those of ordinary skill in the art, these terms as used herein can refer to the combination comprising the mammalian cell population and the medium in which the population is suspended. The terms “media”, “medium”, “cell culture medium”, “culture medium”, “tissue culture medium”, “tissue culture media”, and “growth medium” as used herein refer to a solution containing nutrients which nourish growing cultured eukaryotic cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation. The medium can also be a “defined medium” or “chemically defined medium” which refers to a serum-free medium that contains no proteins, hydrolysates or components of unknown composition. Defined media are free of animal-derived components, and all components have a known chemical structure. One of skill in the art understands a defined medium can comprise recombinant glycoproteins or proteins, for example, but not limited to, hormones, cytokines, interleukins and other signaling molecules. The cell culture medium is generally “serum free” when the medium is essentially free of serum, or fractions thereof, from any mammalian source (e.g. fetal bovine serum (FBS)). By “essentially free” it is meant that the cell culture medium comprises between about 0-5% serum, preferably between about 0-1% serum, and most preferably between about 0-0.1% serum. Advantageously, serum-free “defined” medium can be used, wherein the identity and concentration of each of the components in the medium is known (e.g., an undefined component such as bovine pituitary extract (BPE) is not present in the culture medium). Iron is required by cells to maintain proper cell growth and division. In some instances, the serum-free medium is supplemented with iron. In some instances, the serum free medium is further supplemented with transferrin to reduce the toxic effects of free iron (e.g., generation of reactive oxygen species) (see, e.g., Garcia-Alfonso et al., Changes in antioxidative activities induced by Fe (II) and Fe (III) in cultured Vero cells. 1996. (8661525) Arch Environ Contam Toxicol. 30 (4): p. 431-6). In some instances, an iron chelator can be used in substitution of transferrin to suppress the toxic effect of free iron. In some instances, the iron chelator is citrate (see, e.g., Neumannova et al., Growth of human tumor cell lines in transferrin-free, low-iron medium, In Vitro Cell Dev Biol Anim, 1995 September; 31(8):625-32). In some instances, the serum-free medium is supplemented with ferric citrate as a source of iron and citrate.

The terms “recombinantly expressed protein” and “recombinant protein” and “protein of interest” as used herein, in some instances, refer to a protein expressed from a host cell that has been genetically engineered to express that protein. The recombinantly expressed protein can be identical or similar to proteins that are normally expressed in the host cells. The recombinantly expressed protein can also be foreign to the host cell, e.g., heterologous to peptides normally expressed in the host cell. Alternatively, the recombinantly expressed protein can be chimeric in that portions of the protein contain amino acid sequences that are identical or similar to proteins normally expressed in the host cell, while other portions are foreign to the host cell. In certain instances, the recombinant protein is an antibody, an immunoadhesin, a Transforming Growth Factor (TGF) beta superfamily signaling molecule, a blood clotting factor, a Fc fusion protein, combinations thereof, or fragments thereof. As used herein, the terms “recombinantly expressed protein” and “recombinant protein” and “protein of interest” also encompass an antibody produced by a hybridoma.

In some instances, the recombinant protein is an anti-α-synuclein monoclonal antibody (mAb). In some instances, the anti-α-synuclein monoclonal antibody (mAb) comprises an ATP-binding site and/or binds a target molecule comprising an ATP-binding site. Without wishing to be bound by any particular theory, certain host cell proteins (e.g., HSP90) have been observed to co-purify with recombinantly expressed anti-α-synuclein monoclonal antibody, and these host cell proteins are particularly difficult to remove. For example, it is thought that HSP90 interacts with anti-α-synuclein mAb, and the addition of ATP could potentially release the mAb by driving a conformational change in HSP90 to complete the folding of its substrates. It is also theorized that the Fab region of anti-α-synuclein mAb could bind HSP90 because the mAb recognizes a linear epitope at the N-terminus of α-synuclein at residues 1-10, and other proteins that share a similar sequence could potentially be off target antigens. It is further thought that the interaction could be mediated by α-synuclein because HSP90b has been recently shown to associate with it (see, e.g., Burmann et al., Regulation of α-synuclein by chaperones in mammalian cells, Nature volume 577, pages 127-132(2020)). Further, α-synuclein is highly expressed in human ovarian tissue so it could also be expressed by CHO cells as a host cell protein. In some instances, the modified production conditions of the application (e.g., decreased iron and/or citrate concentration, temperature shifts, carbon filtration) effectively reduce host cell protein impurities (e.g., HSP90, perilipin). Accordingly, the methods described herein can be extended to remove host cell proteins during a recombinant production process.

Perilipin is a protein involved in the regulation of lipid breakdown. Cell lines that exhibit higher lipid oxidation activity may have higher reactive oxygen species (ROS generation). Iron is known to be a source of ROS, and an upregulation of Perilipin in these cells may be a mechanism for the cells to combat ROS. In some instances, reducing free iron in cell culture medium may be a path to reduce ROS, thereby decreasing perilipin expression in host cells.

In certain cases, it can be beneficial or necessary to supplement the cell culture during the growth and/or subsequent production phase with nutrients or other medium components that have been depleted or metabolized by the cells. For example, it might be advantageous to supplement the cell culture with nutrients or other medium components observed to have been depleted. Alternatively or additionally, it can be beneficial or necessary to supplement the cell culture prior to the subsequent production phase. As non-limiting examples, it can be beneficial or necessary to supplement the cell culture with hormones and/or other growth factors, particular ions (such as iron, sodium, chloride, calcium, magnesium, and phosphate), buffers, vitamins, nucleosides or nucleotides, trace elements (inorganic compounds usually present at very low final concentrations), amino acids, lipids, or glucose or other energy source. These supplementary components, including the amino acids, can all be added to the cell culture at one time, or they can be provided to the cell culture in a series of additions. In some instances, the supplementary components are provided to the cell culture at multiple times in proportional amounts. In some instances, it can be desirable to provide only certain of the supplementary components initially, and provide the remaining components at a later time. In yet other instances, the cell culture is fed continually with these supplementary components.

In some instances, the cell culture medium described herein comprises iron and/or citrate. In some instances, the low iron/citrate cell culture medium described herein reduces certain host cell proteins (e.g., heat shock protein 90 (HSP90), and/or perilipin) as compared to a control cell culture medium having a higher concentration of iron and/or citrate.

In some instances, a control cell culture medium comprises iron at a concentration of less than 5 mM, less than 4.5 mM, less than 4 mM, less than 3.5 mM, less than 3 mM, less than 2.5 mM, less than 2 mM, less than 1.5 mM, or less than 1 mM. In some instances, a control cell culture medium comprises iron at a concentration of at least 1 mM, at least 1.5 mM, at least 2 mM, at least 2.5 mM, at least 3 mM, at least 3.5 mM, at least 4 mM, at least 4.5 mM, or at least 5 mM. In some instances, a control cell culture medium comprises iron at any concentration between 4 mM and 5 mM (e.g., 4 mM, 4.1 mM, 4.2 mM, 4.3 mM, 4.4 mM, 4.5 mM, 4.6 mM, 4.7 mM, 4.8 mM, 4.9 mM, or 5 mM), between 3 mM and 4 mM (e.g., 3 mM, 3.1 mM, 3.2 mM, 3.3 mM, 3.4 mM, 3.5 mM, 3.6 mM, 3.7 mM, 3.8 mM, 3.9 mM, or 4 mM), between 2 mM and 3 mM (e.g., 2 mM, 2.1 mM, 2.2 mM, 2.3 mM, 2.4 mM, 2.5 mM, 2.6 mM, 2.7 mM, 2.8 mM, 2.9 mM, or 3 mM), between 1 mM and 3 mM (e.g., 1 mM, 1.1 mM, 1.2 mM, 1.3 mM, 1.4 mM, 1.5 mM, 1.6 mM, 1.7 mM, 1.8 mM, 1.9 mM, or 2 mM), between 2 mM and 5 mM, between 2 mM and 4.5 mM, between 2 mM and 4 mM, between 2 mM and 2.5 mM, between 1 mM and 2 mM, between 2 mM and 2.8 mM, between 2.2 mM and 2.8 mM, between 2.2 mM and 2.6 mM, or between 2.2 mM and 2.5 mM.

In some instances, the cell culture medium described herein comprises a reduced level of iron compared to the control cell culture medium. In some instances, the cell culture medium described herein comprises iron at a concentration up to 95%, up to 90%, up to 85%, up to 80%, up to 75%, up to 70%, up to 65%, up to 60%, up to 55%, up to 50%, up to 45%, up to 40%, up to 35%, up to 30%, up to 25%, up to 20%, up to 15%, up to 10%, or up to 5% of the iron concentration of the control cell culture medium. In some instances, the cell culture medium described herein comprises iron at any concentration between 90% and 99%, between 80% and 90%, between 70% and 90%, between 60% and 70%, between 50% and 60%, between 40% and 50%, between 30% and 40%, between 20% and 30%, between 10% and 20%, between 1% and 10%, between 99% and 50%, between 99% and 60%, between 99% and 70%, between 99% and 80%, between 80% and 50%, between 70% and 50%, between 60% and 50%, between 60% and 55%, between 60% and 40%, between 55% and 40%, or between 50% and 40% of iron concentration of the control cell culture medium. In some instances, the cell culture described herein comprises iron at a concentration that is 50% of the iron concentration of the control cell medium.

In some instances, the cell culture described herein comprises iron at a concentration less than 2.5 mM, less than 2 mM, less than 1.5 mM, less than 1.2 mM, less than 1 mM, less than 900 μM, less than 800 μM, less than 700 μM, less than 600 μM, less than 500 M, less than 400 μM, less than 300 μM, less than 200 μM, less than 100 μM, less than 90 μM, less than 80 μM, less than 70 μM, less than 60 μM, less than 50 μM, less than 40 μM, less than M, less than 20 μM, or less than 10 μM. In some instances, the cell culture medium described herein comprises iron at any concentration between about 100 μM and about 2500 M, between about 200 μM and about 2400 μM, between about 300 μM and about 2300 μM, between about 400 μM and about 2200 μM, between about 500 μM and about 2100 μM, between about 600 μM and about 2000 μM, between about 700 μM and about 1900 μM, between about 800 μM and about 1800 μM, between about 900 μM and about 1700 μM, between about 1000 μM and about 1600 μM, between about 1100 μM and about 1500 μM, between about 1200 μM and about 1400 μM, between about 1400 μM and about 1500 μM, between about 500 μM and about 1500 μM, between about 600 μM and about 1500 μM, between about 700 μM and about 1500 μM, between about 700 μM and about 1500 μM, between about 800 μM and about 1500 μM, between about 900 μM and about 1500 μM, between about 1000 μM and about 1500 μM, between about 1100 μM and about 1500 μM, between about 1200 μM and about 1500 μM, between about 1300 μM and about 1500 μM, between about 700 μM and about 1400 μM, between about 700 μM and about 1300 μM, between about 700 μM and about 1200 μM, between about 700 μM and about 1100 μM, between about 700 μM and about 1000 μM, between about 700 μM and about 900 μM, between about 700 μM and about 800 μM, between about 1000 μM and about 1100 μM, between about 1000 μM and about 1200 μM, between about 1000 μM and about 1300 μM, between about 1100 M and about 1400 μM, between about 1000 μM and about 1400 μM, between about 1000 μM and about 1500 μM, between about 1000 μM and about 1600 μM, between about 1000 μM and about 1700 μM, between about 1000 μM and about 1800 μM, between about 1000 μM and about 1900 μM, between about 1000 μM and about 2000 μM, between about 1000 μM and about 2100 μM, between about 1000 μM and about 2200 μM, between about 1000 μM and about 2300 μM, between about 1000 μM and about 2400 μM, or between about 1000 μM and about 2500 μM. In some instances, the cell culture medium described herein comprises iron at a concentration of 1083 μM.

In some instances, the iron concentration at the start of the cell culture (e.g., day 0 of the cell culture after the cells are thawed) is less than 1500 μM, less than 1400 μM, less than 1300 μM, less than 1200 μM, less than 1100 μM, less than 1000 μM, less than 900 μM, or less than 800 μM. In some instances, the iron concentration at the start of the culture (e.g., day 0 of the cell culture after the cells are thawed) is between about 500 μM and about 1500 μM, between about 600 μM and about 1400 μM, between about 700 μM and about 1300 μM, between about 800 μM and about 1200 μM, between about 900 μM and about 1100 μM, between about 1000 μM and about 1200 μM, or between about 1000 μM and about 1100 μM. In some instances, the iron concentration at the start of the cell culture (e.g., day 0 of the cell culture after the cells are thawed out) is 1083 μM. In some instances, the iron concentration is maintained in the cell culture during production phase (e.g., days 3-14 of the cell culture) at any concentration between about 500 μM and about 2100 μM, between about 600 μM and about 2000 μM, between about 700 μM and about 1900 μM, between about 800 μM and about 1800 M, between about 900 μM and about 1700 μM, between about 500 μM and about 1500 μM, between about 600 μM and about 1500 μM, between about 700 μM and about 1500 μM, between about 800 μM and about 1500 μM, between about 900 μM and about 1500 μM, between about 700 μM and about 1300 μM, between about 700 μM and about 1200 μM, between about 700 μM and about 1100 μM, between about 700 μM and about 1000 μM, between about 700 μM and about 900 μM, or between about 700 μM and about 800 μM. In some instances, the iron concentration is maintained in the cell culture during production phase (e.g., days 3-14 of the cell culture) at any concentration between about 700 μM and about 1000 M. In some embodiments, the iron concentration at the start of the cell culture (e.g., Day 0 cell culture after the cells are thawed out) is 1083 μM and is maintained in the cell culture during production phase (e.g., days 3-14 of the cell culture) at any concentration between about 700 μM and about 1000 μM.

Alternatively or in addition, in some instances, a control cell culture medium comprises citrate at a concentration of less than 18 mM, less than 17 mM, less than 16 mM, less than 15 mM, less than 14 mM, less than 13 mM, less than 12 mM, less than 11 mM, less than 10 mM, less than 9 mM, less than 8.5 mM, less than 8 mM, less than 7.5 mM, less than 7 mM, less than 6.5 mM, less than 6 mM, less than 5.5 mM, less than 5 mM, less than 4.5 mM, less than 4 mM, less than 3.5 mM, less than 3 mM, less than 2.5 mM, less than 2 mM, less than 1.5 mM, or less than 1 mM. In some instances, a control cell culture medium comprises citrate at any concentration between 18 mM and 5 mM, between 18 mM and 6 mM, between 18 mM and 7 mM, between 18 mM and 8 mM, between 18 mM and 9 mM, between 18 mM and 10 mM, between 18 mM and 11 mM, between 18 mM and 12 mM, between 18 mM and 13 mM, between 18 mM and 14 mM, between 18 mM and 15 mM, between 18 mM and 16 mM, between 18 mM and 17 mM, between 8 mM and 1 mM, between 7 mM and 2 mM, between 6 mM and 3 mM, between 5 mM and 4 mM, between 5 mM and 3 mM, between 5 mM and 2 mM, or between 5 mM and 1 mM.

In some instances, the cell culture medium described herein comprises reduced level of citrate compared to the control cell culture medium. In some instances, the cell culture medium described herein comprises citrate at a concentration up to 95%, up to 90%, up to 85%, up to 80%, up to 75%, up to 70%, up to 65%, up to 60%, up to 55%, up to 50%, up to 45%, up to 40%, up to 35%, up to 30%, up to 25%, up to 20%, up to 15%, up to 10%, or up to 5% of the citrate concentration of the control cell culture medium. In some instances, the cell culture medium described herein comprises citrate at any concentration between 90% and 99%, between 80% and 90%, between 70% and 90%, between 60% and 70%, between 50% and 60%, between 40% and 50%, between 30% and 40%, between 20% and 30%, between 10% and 20%, between 1% and 10%, between 99% and 50%, between 99% and 60%, between 99% and 70%, between 99% and 80%, between 80% and 50%, between 70% and 50%, between 60% and 50%, between 60% and 55%, between 60% and 40%, between 55% and 40%, or between 50% and 40% of citrate concentration of the control cell medium. In some instances, the cell culture described herein comprises citrate at a concentration that is 50% of the citrate concentration of the control cell medium.

In some instances, the cell culture described herein comprises citrate at a concentration less than 10 mM, less than 9 mM, less than 8.5 mM, less than 8 mM, less than 7 mM, less than 6 mM, less than 5 mM, less than 4 mM, less than 3 mM, or less than 2 mM. In some instances, the ratio of the citrate concentration to the iron concentration in the cell culture medium is about 1.5:1, about 1.6:1, about 1.7:1, about 1.8:1, about 1.9:1, about 2:1, about 2.1:1, about 2.2:1, about 2.3:1, about 2.4:1, or about 2.5:1. In some instances, the ratio of the citrate concentration to the iron concentration in the cell culture medium is about 2:1. In some instances, the cell culture medium described herein comprises citrate at any concentration between about 1 mM and about 10 mM, between about 1 mM and about 9 mM, between about 1 mM and about 8 mM, between about 1 mM and about 7 mM, between about 1 mM and about 6 mM, between about 1 mM and about 5 mM, between about 1 mM and about 4 mM, between about 1 mM and about 3 mM, between about 1 mM and about 2 mM, between about 1.2 mM and about 5 mM, between about 1.4 mM and about 4 mM, between about 1.6 mM and about 3 mM, between about 1.4 mM and about 2.5 mM, between about 1.4 mM and about 2.2 mM, between about 1.4 mM and about 2 mM, between about 1.6 mM and about 3 mM, between about 1.8 mM and about 3 mM, between about 1.8 mM and about 2.5 mM, between about 2 mM and about 2.5 mM, or between about 2.1 mM and about 2.2 mM. In some instances, the cell culture described herein comprises citrate at a concentration of 2.164 mM.

In some instances, the citrate concentration at the start of the cell culture (e.g., day 0 of the cell culture after the cells are thawed) is less than 5 mM, less than 4 mM, less than 3 mM, or less than 2 mM. In some instances, the citrate concentration at the start of the cell culture (e.g., day 0 of the cell culture after the cells are thawed) is between about 1 mM and about 5 mM, between about 1 mM and about 4 mM, between about 1 mM and about 3 mM, between about 1 mM and about 2 mM, between about 2 mM and about 5 mM, between about 2 mM and about 4 mM, between about 2 mM and about 3 mM, or between about 2 mM and about 2.5 mM. In some instances, citrate is not present at the start of the culture (e.g., day 0 of the cell culture after the cells are thawed), and is added to the culture in any of the subsequent days (e.g., day 1, day 2, day 3, day 4, day 5, day 6, day 7, day 8, day 9, day 10, day 11, day 12, day 13, day 14, day 15 or further) of the cell culture to reach any of the citrate concentrations described herein. Accordingly, in some instances, the citrate concentration at the start of the cell culture (e.g., day 0 of the cell culture after the cells are thawed) is less than 1 mM (e.g., between about 0 mM and about 1 mM, between about 0 mM and about 0.75 mM, between about 0 mM and about 0.5 mM, between about 0 mM and about 0.25 mM, or between about 0 mM and about 0.1 mM). In some instances, the citrate concentration at the start of the cell culture (e.g., day 0 of the cell culture after the cells are thawed out) is 2.164 mM. In some instances, the citrate concentration is maintained in the cell culture during production phase (e.g., days 3-14 of the cell culture) at any concentration between about 1 mM and about 10 mM, between about 1 mM and about 9 mM, between about 1 mM and about 8 mM, between about 1 mM and about 7 mM, between about 1 mM and about 6 mM, between about 1 mM and about 5 mM, between about 1 mM and about 4 mM, between about 1 mM and about 3 mM, between about 1 mM and about 2 mM, between about 1.2 mM and about 5 mM, between about 1.4 mM and about 4 mM, between about 1.6 mM and about 3 mM, between about 1.4 mM and about 2.5 mM, between about 1.4 mM and about 2.2 mM, between about 1.4 mM and about 2 mM, between about 1.6 mM and about 3 mM, between about 1.8 mM and about 3 mM, between about 1.8 mM and about 2.5 mM, between about 2 mM and about 2.5 mM, or between about 2.1 mM and about 2.2 mM. In some instances, the citrate concentration is maintained in the cell culture during production phase (e.g., days 3-14 of the cell culture) at any concentration between about 1.4 mM and about 2 mM. In some instances, the citrate concentration at the start of the cell culture (e.g., Day 0 cell culture after the cells are thawed out) is 2.164 μM and is maintained in the cell culture during production phase (e.g., days 3-14 of the cell culture) at any concentration between about 1.4 mM and about 2 mM.

In some instances, the iron is added to the cell culture media in the form of an iron salt (e.g., ferric salt or ferrous salt) to reach an iron concentration described herein. In some instances, the iron is added to the cell culture in the form of ferric chloride to reach an iron concentration described herein. In some instances, the iron is added to the cell culture media in the form of a more concentrated stock solution of ferric chloride (e.g., an added volume from a stock solution of ferric chloride that is 2×, 3×, 4×, 5×, 6×, 7×, 8×, 9×, 10×, 15×, 20× or more concentrated than the desired iron concentration in the cell culture media). In some instances, the citrate is added to the cell culture media in the form of a citrate salt (e.g., sodium citrate) to reach a citrate concentration described herein. In some instances, the citrate is added to the cell culture media in the form of a more concentrated stock solution of sodium citrate (e.g., an added volume of a stock solution of sodium citrate that is 2×, 3×, 4×, 5×, 6×, 7×, 8×, 9×, 10×, 15×, 20× or more concentrated than the desired citrate concentration in the cell culture media). In some instances, the stock solution is prepared by adding ferric chloride and/or sodium citrate to an aqueous solution in an amount such that the iron and citrate solution is 2×, 3×, 4×, 5×, 6×, 7×, 8×, 9×, 10×, 15×, 20× or more concentrated than the desired iron and/or citrate concentration in the cell culture media. In some instances, a volume of the iron and citrate stock solution is added to a bulk cell culture media in an amount to reach the iron and citrate concentration described herein in the cell culture media. The stock solution can be prepared and used in accordance with known methods in the art.

In some instances, the host cells are cultured in the cell culture medium with reduced iron and/or reduced citrate concentrations described herein throughout more than one stage of the recombinant production process (e.g., growth phase through production phase). In some instances, the host cell are cultured in the cell culture medium with reduced iron and/or reduced citrate concentrations described herein at a specific stage of the recombinant production process (e.g., growth phase or production phase). In some instances, the iron and/or citrate concentration is maintained throughout the cell culture via having sufficient amounts carried over from one stage to the next (e.g., having sufficient amount carried over from the growth phase to the production phase such that there is no need to further supplement iron and/or citrate in the production phase). In some instances, the iron and/or citrate concentration is maintained throughout the cell culture by supplementing the culture with additional iron and/or citrate.

In some instances, the host cells are cultured in the cell culture medium with iron and/or citrate at a concentration described herein at the start of the growth phase. For example, in some instances, the cells are thawed and added to a cell culture medium containing iron and/or citrate at a concentration described herein. In other instances, the cells are thawed and added to a cell culture medium that does not contain iron and/or citrate, and the cell culture is then supplemented with iron and/or citrate (e.g., by directly adding iron and/or citrate in powder form or by adding a volume of an iron and/or citrate stock solution described herein) to reach an iron and/or citrate concentration described herein. In some instances, the cells are then cultured in the cell culture media with an iron and/or citrate concentration described herein throughout the growth phase (e.g., day 0, day 1, and day 2 of the cell culture). In some instances, the cells are cultured in the cell culture media with an iron and/or citrate concentration described herein throughout the growth phase (e.g., day 0, day 1, and day 2 of the cell culture) and the production phase (e.g., day 3-14 of the cell culture). In some instances, the iron and/or citrate are added to the cell culture as a supplemental bolus (e.g., a bolus of iron and/or citrate in a form described herein is added to the cell culture medium at certain days of the cell culture process). In some instances, the iron and/or citrate are added to the cell culture medium in a continuous feeding process (e.g., iron and/or citrate in a form described herein is continuously fed to the cell culture medium throughout the cell culture process).

In some instances, the iron and/or citrate concentration is monitored throughout the growth phase and/or the production phase of the cell culture. The iron and/or citrate concentration can be measured by any known methods in the art. In some instances, the iron and/or citrate concentration in the cell culture is measured off-line (e.g., using Raman Spectroscopy). In some instances, the iron and/or citrate concentration in the cell culture is measured at-line (e.g., using Raman Spectroscopy). In some instances, the iron and/or citrate concentration in the cell culture is measured on-line (e.g., using Raman Spectroscopy). In some instances, the iron and/or citrate concentration in the cell culture is measured in-line (e.g., using Raman Spectroscopy). The use of Raman spectroscopy in certain aspects of Biopharma manufacturing is previously described, e.g., WO2020238918; and Buckley et al., Applications of Raman Spectroscopy in Biopharmaceutical Manufacturing: A Short Review, Applied Spectroscopy, Volume: 71 issue: 6, page(s): 1085-1116, the entire contents of each of which are incorporated herein by reference. In some instances, citrate concentration is measured in-line (e.g., using Raman Spectroscopy), and iron concentration is measured at-line or off-line, for example, by other suitable methods in the art (e.g., mass spectrometry, membrane-based methods such as the method described in Zamani et al., Monitoring of iron (III) ions with a Fe³⁺-PVC membrane sensor based on 4, 4′-Dimethoxybenzil bisthiosemicarbazone, Journal of the Chilean Chemical Society, 54(2), June 2009, or chemistry-based methods such as the method described by Hirayama et al., Chemical tools for detecting Fe ions, J Clin Biochem Nutr, 2017 January; 60(1):39-48). In some instances, both citrate and iron are measured by any suitable methods used in the art or described herein.

In some instances, the iron and/or citrate concentration in the cell culture is maintained by supplementing the cell culture with iron and/or citrate (e.g., either alone or with other components of the cell culture medium) in response to the monitoring results. In some instances, the cell culture is supplemented manually with iron and/or citrate. In some instances, the cell culture is supplemented automatically with iron and/or citrate. In some instances, the cell culture is a fed-batch culture and the cell cultured is supplemented with iron and/or citrate as described elsewhere herein when the iron and/or citrate concentration is lower than the desired concentration described herein (e.g., by automatically monitoring and supplementing). In some instances, the cell culture is a perfusion culture which circulates medium through a growing culture, allowing constant supplementation of iron and/or citrate. In some instances, only iron concentration in the cell culture is monitored.

In some instances, regulating the levels of iron and/or citrate in a cell culture during the recombinant production process can reduce the host cell protein (e.g., HSP90 and/or perilipin). In some instances, decreasing the second temperature of a production stage temperature shift in a cell culture during the recombinant production process can reduce the host cell protein (e.g., HSP90 and/or perilipin). In some instances, the present application provides a method comprising culturing host cells expressing a recombinant protein in a cell culture medium at a first temperature; and decreasing the first temperature to a second temperature. In some instances, the first temperature of the cell culture can be selected based on the temperatures or range of temperatures at which the cell culture remains viable, at which a high level of protein is produced, at which production or accumulation of metabolic waste products is minimized, and/or any combination of these or other factors deemed important. In general, most mammalian cells grow well and/or can produce high levels or protein within a range of about 25° C. to 42° C. In certain instances, the cell culture is grown at a first temperature of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C. In some instances, the first temperature of the cell culture is between about 32° C. and 36° C., between about 33° C. and 35° C., between about 34° C. and 36° C., between about 30° C. and 38° C., between about 31° C. and 37° C., or between about 35° C. and 37° C. In some instances, the first temperature of the cell culture is 35° C. In some instances, the decrease of the first temperature to the second temperature occurs after the growth phase of the cell culture. “Growth phase” of the cell culture, in some instances, refers to the period of exponential cell growth (the log phase) where cells are generally rapidly dividing. During this phase, cells are cultured for a period of time, usually between 1-4 days, and under such conditions that cell growth is maximized. The determination of the growth cycle for the host cell can be determined for the particular host cell envisioned without undue experimentation. “Period of time and under such conditions that cell growth is maximized” and the like, refer to those culture conditions that, for a particular cell line, are determined to be optimal for cell growth and division. During the growth phase, cells are cultured in nutrient medium containing the necessary additives generally at about 25-42° C., in a humidified, controlled atmosphere, such that optimal growth is achieved for the particular cell line. Cells are maintained in the growth phase for a period of about between one and seven days, e.g., between two to six days, e.g., six days. The length of the growth phase for the particular cells can be determined without undue experimentation. For example, the length of the growth phase will be the period of time sufficient to allow the particular cells to reproduce to a viable cell density within a range of about 20%-80% of the maximal possible viable cell density if the culture was maintained under the growth conditions.

In some instances, the decrease of the first temperature to the second temperature occurs during the production phase of the cell culture. “Production phase” or “protein production phase” of the cell culture, in some instances, refers to the period of time during which cell growth has plateaued. During the production phase, logarithmic cell growth has ended and protein production is primary. During this period of time, the medium is generally supplemented to support continued protein production and to achieve the desired protein product. The production phase is typically between about three and about fourteen days, e.g., between about five and about eight days, e.g., six days. In some instances, the decrease of the first temperature to the second temperature occurs on day 1, day 2, day 3, day 4, day 5, day 6, day 7, day 8, day 9, day 10, day 11, day 12, day 13, day 14, day 15, day 16, day 17, or day 18 of the production phase of the cell culture. In some instances, the decrease of the first temperature to the second temperature occurs on day 3 of the production phase. In some instances, the first temperature is decreased by 1° C., 2° C., 3° C., 4° C., 5° C., 6° C., 7° C., 8° C., 9° C., or 10° C. In some instances, the first temperature is 35° C. and it is decreased by at least 1, at least 2, at least 3, at least 4, at least 5, at least 6, at least 7, or at least 8° C. to reach the second temperature. In some instances, the first temperature is 35° C. and it is decreased by at least 5° C. to reach the second temperature. In some instances, the second temperature is lower than 33° C., 32° C., 31° C., 30° C., 29° C., 28° C., 27° C., or 26° C. In some instances, the second temperature is lower than 31° C. In some instances, the second temperature of the cell culture is between about 28° C. and 30° C., between about 25° C. and 30° C., between about 25° C. and 32° C., between about 25° C. and 33° C., between about 28° C. and 32° C., or between about 27° C. and 31° C. In some instances, the second temperature is 29° C. In some instances, the temperature shift can occur more than once and does not need to necessarily be a decrease from a current temperature. Moreover, the transition phase and the temperature shift can occur on any day during the production run, although most methods of production include multi-phase processes, or a single-phase process.

When shifting the temperature of the culture, the temperature shift may be relatively gradual. For example, it may take several hours or days to complete the temperature change. In some instances, a first temperature is decreased to a second temperature over a period that is at least 6 hours and up to 72 hours (e.g., at least 6 hours and up to 24 hours, at least 12 hours and up to 24 hours, at least 24 hours and up to 48 hours). Alternatively, the temperature shift may be relatively abrupt. In some instances, a first temperature is decreased to a second temperature over a period that is less than about 12 hours (e.g., less than about 6 hours, less than about 3 hours, less than about 1 hour, less than about 30 minutes, at least 1 hour and up to 6 hours, at least 1 minute and up to 60 minutes). The temperature may be steadily increased or decreased during the culture process. Alternatively, the temperature may be increased or decreased by discrete amounts at various times during the culture process. The second temperature(s) or temperature range(s) may be lower than or higher than the first temperature.

In some instances, the low iron, low citrate and/or low temperature condition does not affect cell viability, viable cell density, and/or production titer of the recombinant protein. The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time. The term “titer” as used herein refers to the total amount of recombinantly expressed glycoprotein or protein produced by a cell culture divided by a given amount of medium volume. Titer is typically expressed in units of milligrams of glycoprotein or protein per milliliter of medium or in units of grams of glycoprotein or protein per liter of medium.

### II. Regulation of Downstream Purification Process

In some instances, the host cell proteins (e.g., HSP90 and/or perilipin) are reduced during an upstream production process (e.g., cell culture stage, such as a cell growth and/or protein expression phase), and can be further removed by regulating a downstream recombinant protein production process, such as a protein purification stage.

In some instances, the methods provided herein further comprise purifying the recombinant protein by carbon depth filtration. The term “depth filtration” refers to a filtration method using multiple layers of media, or a thicker media that forms a path to retain particles throughout the medium. The term “carbon depth filtration” refers to a filtration method using activated carbon depth filters. Activated carbon is highly porous over a broad range of pore sizes, from cracks and crevices to molecular dimensions. It is this porosity that provides activated carbon's unique adsorptive properties. Generally, activated carbon filter sheets contain surface areas in the range of 500-2000 m²/gm. “Activation” refers to the development of the adsorption properties of carbon. Adsorption occurs when the organic molecules bond to the internal pores of the activated carbon filter sheets. Non-limiting examples of carbon depth filters include Millistak+® Pod, MICRO-CLEAR™ ACTIVATED CARBON DEPTH FILTER MEDIA, ErtelAlsop MicroClear Activated Carbon Depth Filter Sheets, or Depth Filtration BECODISC®.

In some instances, the purification process comprises various types of purification methods (e.g., Protein A chromatography, anion exchange chromatography, cation exchange chromatography, and/or hydrophobic interaction chromatography). In some instances, any of these chromatographic methods can be used in any order to purify the recombinant protein. In some instances, the carbon filter depth filtration can be performed in between any other chromatographic method (e.g., between Protein A chromatography and anion exchange chromatography). The terms “Protein A” and “ProA” are used interchangeably herein and encompass Protein A recovered from a native source thereof, Protein A produced synthetically (e.g. by peptide synthesis or by recombinant techniques), and variants thereof which retain the ability to bind proteins which have a CH2/CH3 region, such as an Fc region. Protein A can be purchased commercially from Repligen, Pharmacia and Fermatech. Protein A is generally immobilized on a solid phase support material. The term “ProA” also refers to an affinity chromatography resin or column containing chromatographic solid support matrix to which is covalently attached Protein A. The term “chromatography” refers to the process by which a solute of interest in a mixture is separated from other solutes in a mixture as a result of differences in rates at which the individual solutes of the mixture migrate through a stationary medium under the influence of a moving phase, or in bind and elute processes. The term “anion exchange resin” is used herein to refer to a solid phase which is positively charged, e.g. having one or more positively charged ligands, such as quaternary amino groups, attached thereto. Commercially available anion exchange resins include DEAE cellulose, QAE SEPHADEX™ and FAST Q SEPHAROSE™ (Pharmacia). A “cation exchange resin” refers to a solid phase which is negatively charged, and which thus has free cations for exchange with cations in an aqueous solution passed over or through the solid phase. A negatively charged ligand attached to the solid phase to form the cation exchange resin may, e.g., be a carboxylate or sulfonate. Commercially available cation exchange resins include carboxy-methyl-cellulose, sulphopropyl (SP) immobilized on agarose (e.g. SP-SEPHAROSE FAST FLOW™ or SP-SEPHAROSE HIGH PERFORMANCE™, from Pharmacia) and sulphonyl immobilized on agarose (e.g. S-SEPHAROSE FAST FLOW™ from Pharmacia). A “mixed mode ion exchange resin” refers to a solid phase which is covalently modified with cationic, anionic, and hydrophobic moieties. A commercially available mixed mode ion exchange resin is BAKERBOND ABX™ (J. T. Baker, Phillipsburg, N.J.) containing weak cation exchange groups, a low concentration of anion exchange groups, and hydrophobic ligands attached to a silica gel solid phase support matrix.

In some instances, the efficiency of carbon depth filtration in removing host cell proteins (e.g., HSP90 and/or perilipin) can be affected by various conditions of the carbon depth filtration (e.g., loading mass, pH, and/or salt concentration). In some instances, the loading mass of the carbon depth filtration effective in removing the host cell protein (e.g., HSP90 and/or perilipin) is less than 4000 g/m², less than 4000 g/m², less than 4000 g/m², less than 4000 g/m², less than 4000 g/m², less than 3500 g/m², less than 3000 g/m², less than 2500 g/m², less than 2000 g/m², less than 1500 g/m², or less than 1000 g/m². In some instances, the loading mass of the carbon depth filtration effective in removing the host cell protein (e.g., HSP90 and/or perilipin) is between about 1000 g/m² and about 4000 g/m², between about 1500 g/m² and about 4000 g/m², between about 2000 g/m² and about 4000 g/m², between about 2500 g/m² and about 4000 g/m², between about 3000 g/m² and about 4000 g/m², between about 3500 g/m² and about 4000 g/m², between about 3800 g/m² and about 4000 g/m², between about 2500 g/m² and about 3500 g/m², between about 2800 g/m² and about 3600 g/m², or between about 3000 g/m² and about 3500 g/m². In some instances, the loading mass of the carbon depth filtration is 3500 g/m². In some instances, the loading mass of the carbon depth filtration is 2500 g/m².

In some instances, the pH of the filtration buffer of the carbon depth filtration effective in removing the host cell protein (e.g., HSP90 and/or perilipin) is between about 5 and about 8, between about 5.5 and about 8, between about 6 and about 8, between about 6.5 and about 8, between about 7 and about 8, between about 7.5 and about 8, between about 7 and about 7.5 (e.g., 7.1, 7.2, 7.3, 7.4, 7.5), between about 6 and about 7, or between about 5 and about 6. In some instances, the pH of the filtration buffer is 7. In some instances, the pH of the filtration buffer is 7.3.

In some instances, the salt (e.g., sodium chloride) concentration in the filtration buffer of the carbon depth filtration effective in removing the host cell protein (e.g., HSP90 and/or perilipin) is between about 20 mM and about 100 mM, between about 30 mM and about 90 mM, between about 40 mM and about 80 mM, between about 50 mM and about 70 mM, between about 50 mM and about 80 mM, between about 50 mM and about 90 mM, between about 50 mM and about 100 mM, between about 60 mM and about 80 mM, between about 60 mM and about 100 mM, between about 20 mM and about 30 mM, between about 20 mM and about 40 mM, between about 20 mM and about 50 mM, between about 30 mM and about 40 mM, between about 40 mM and about 50 mM, or between about 30 mM and about 50 mM. In some instances, the salt (e.g., sodium chloride) is at a concentration of 25 mM in the filtration buffer. In some instances, the salt (e.g., sodium chloride) is at a concentration of 50 mM in the filtration buffer.

In some instances, the methods provided herein comprise a combination of modulated upstream cell culture conditions (e.g., low temperature, low iron and/or low citrate) and downstream purification process conditions (e.g., carbon depth filtration) to reduce host cell proteins (e.g., HSP90 and/or perilipin). In some instances, the method can reduce the host cell proteins (e.g., HSP90 and/or perilipin) by at least 10%, at least 20%, at least 30%, at least 40%, at least 50%, at least 60%, at least 70%, at least 80%, at least 90%, at least 95%, at least 96%, at least 97%, at least 98%, at least 99%, or 100% compared to a control recombinant protein production process (e.g., a method that uses cell culture medium having higher temperature, higher iron, and/or higher citrate concentrations, and/or a method that does not perform carbon depth filtration).

The foregoing description is to be understood as being representative only and is not intended to be limiting. Alternative methods and materials for implementing the disclosure and also additional applications will be apparent to one of skill in the art, and are intended to be included within the accompanying claims.

### EXAMPLES

### Example 1. Identification of Unique Host Cell Impurities During Recombinant Protein Production

Host cell proteins (HCPs) are process-related impurities associated with the production of recombinant proteins. These impurities can negatively impact the stability, safety, and efficacy of the therapeutic protein and can be challenging to remove. After the production phase, the harvest cell culture fluid (HCCF) is typically processed through a series of filtration and chromatographic unit operations. These unit operations are designed to purify the protein of interest by removing unwanted impurities such as HCPs.

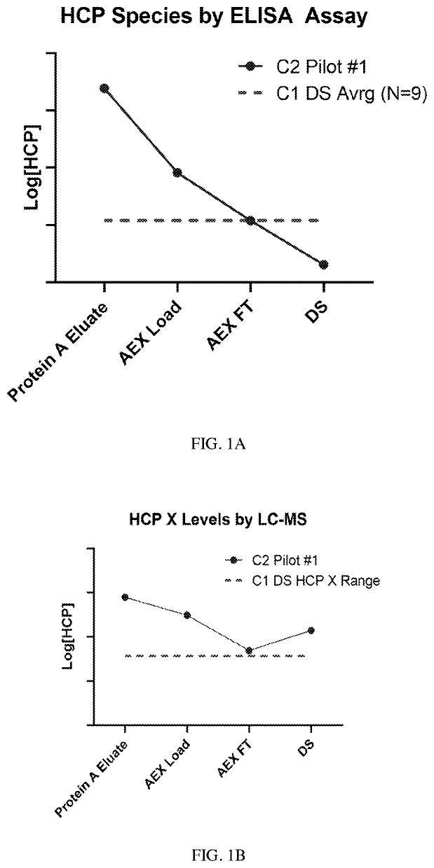

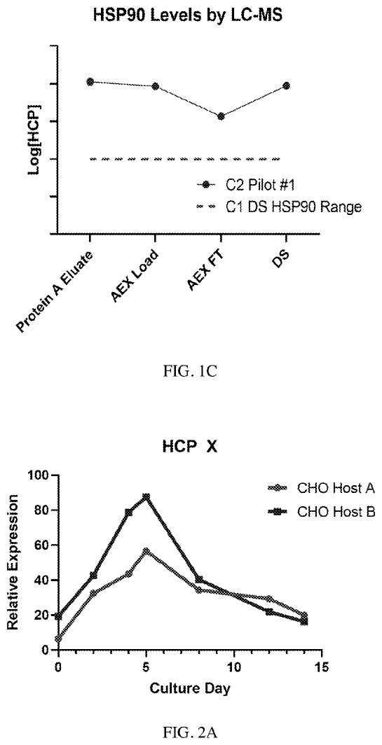

During development of the production process of a recombinant antibody, a new host cell line (CHO cell host B) was used. By comparing the HCP produced by CHO cell host B to the original cell line (CHO cell host A) used to produce the recombinant antibody, it was found that, while the total HCP species in the drug substance (DS) were comparable between the two cell lines (FIG. 1A), two HCPs were present at high levels in the drug substance produced by CHO cell host B (FIGS. 1B and 1C). The two HCPs were later identified by LC-MS as heat shock protein 90 (HSP90) and perilipin.

### Example 2: Upstream Process to Reduce Host Cell Proteins

It is possible to regulate both the upstream process (cell culture) and downstream purification process to reduce/remove the quantity of HCPs present in the drug substance.

In order to regulate the upstream process to reduce the expression of the HCPs in HCCF, studies were done to investigate the cellular pathways in which HSP90 and Perilipin are involved.

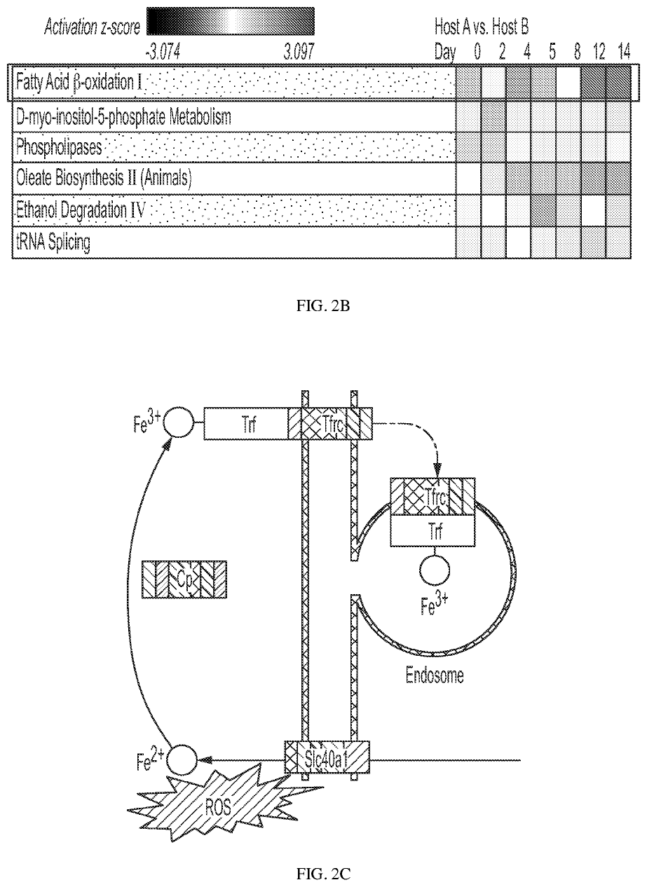

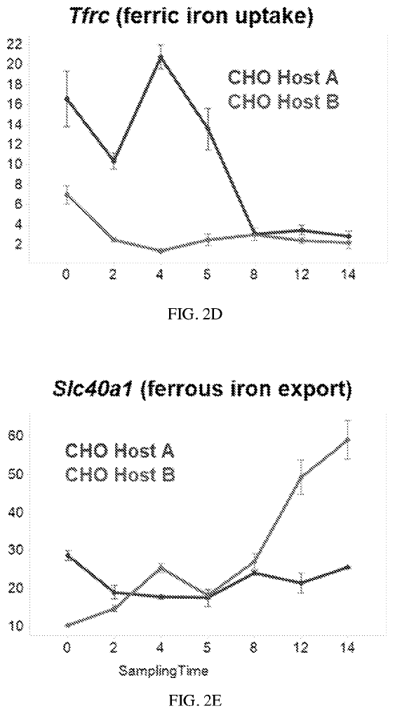

HSP90 is a chaperone protein that is upregulated under heat and/or physiological stress to help with protein folding and degradation. It was hypothesized that decreasing the cell culture temperature may lower HSP90 levels. Perilipin is a protein involved in regulation of lipid breakdown. It was observed that perilipin is expressed at a higher lever in CHO host cell B (FIG. 2A), which is consistent with the observation that CHO host cell B exhibits higher lipid oxidation activity (FIG. 2B). Higher lipid oxidation is correlated to higher energy generation and reactive oxygen species (ROS) generation. It is known that iron is a source of ROS (FIG. 2C), and CHO host cell B exhibited lower iron update (FIG. 2D) and higher iron export (FIG. 2E), which led to more free iron in the culture medium. This data suggests that iron overload occurs in CHO host cell B, which can lead to ROS generation. Therefore, while not wishing to be bound to any particular theory, the upregulation of perilipin may be a mechanism for the cell to combat ROS. It is possible that lowering iron and citrate level in cell medium of CHO host cell B reduces ROS generation, thereby decreasing perilipin expression.

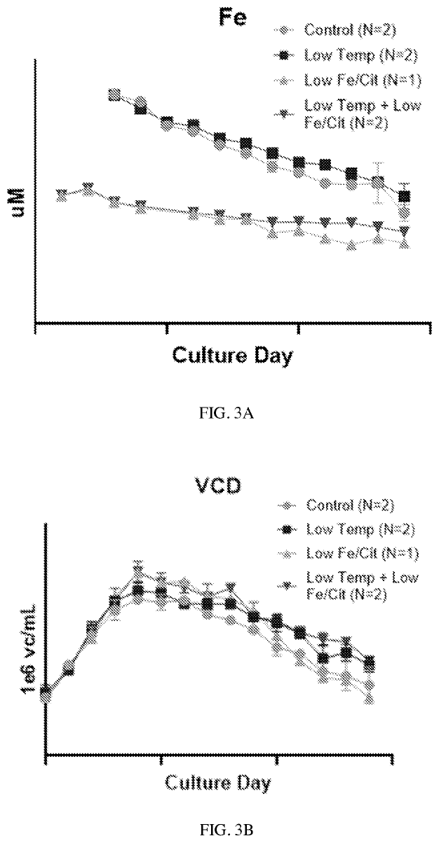

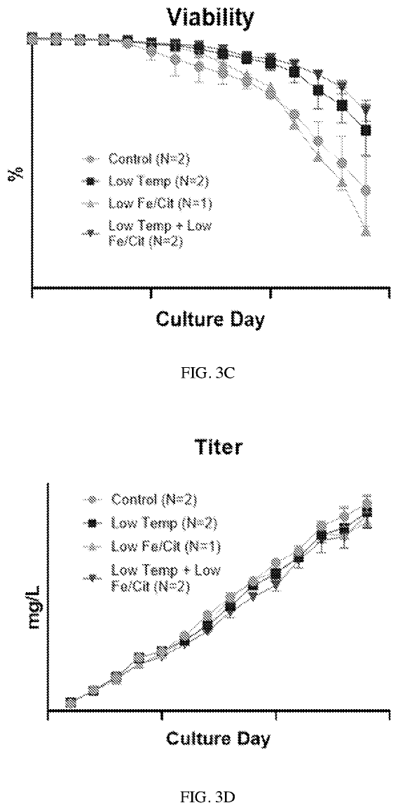

To ensure low temperature and/or low iron/citrate level would not negatively impact cell growth and recombinant antibody production, CHO host cell B expressing a recombinant antibody were cultured under the following conditions: (i) regular medium as control; (ii) low temperate condition; (iii) low iron/citrate condition; and (iv) low temperature/iron/citrate condition. Subsequently, free iron level in the cell medium, viable cell density (VCD), cell viability and antibody titer were evaluated. The results showed the low iron/citrate, and low temperature/iron/citrate condition lowered free iron level in the cell medium (FIG. 3A). VCD, cell viability, and protein titer are comparable to the control group in all experimental groups (FIGS. 3B-3D).

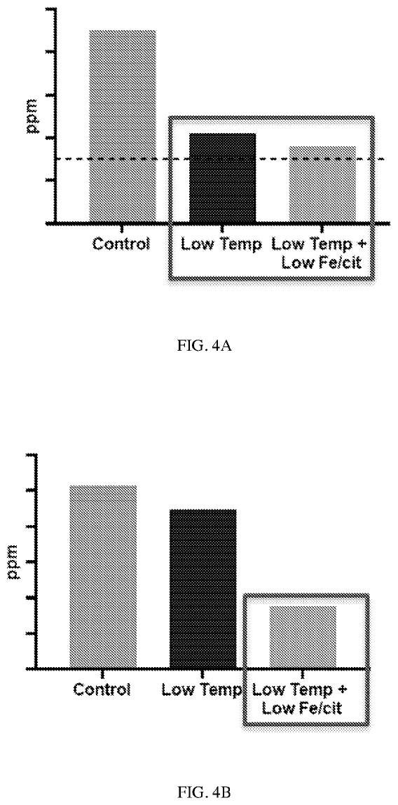

Previously, CHO host cell B were cultured in cell medium containing 2166 μM of iron and 4328 μM citrate throughout the passage phase and production phase. The cells were initially cultured at 35° C. at passage phase. Three days after cells were transitioned into production phase, the culture temperature was decreased to 31° C. In order to test the effect of low temperature and/or low iron/citrate in cell culture medium on HSP90 and perilipin expression, CHO host cell B expressing a recombinant antibody were cultured in either low temperature condition, or low temperature/iron/citrate condition. Under the low temperature condition, the cell culture medium remains the same as previously used. The cell culture was initiated at 35° C. during the passage phase. The culture temperature was decreased to 29° C. three days after the cells were transitioned into production phase and maintained at 29° C. through the production phase. Under the low temperature/iron/citrate condition, the cells were cultured in culture medium containing 1083 μM of iron and 2164 μM citrate throughout the passage phase and production phase. The same temperature shift was performed as the low temperature only condition. The HCCF was harvested and subjected to Protein A chromatography. The elute of Protein A chromatography were tested for the level of HSP90 and perilipin. The results showed that reducing temperature in the upstream cell culture process reduces HSP90 levels intermediate Protein A (ProA) eluate (FIG. 4A), and that reduction of iron and citrate in the cell culture medium can reduce perilipin in the ProA eluate (FIG. 4B).

### Example 3: Downstream Process to Further Remove Host Cell Proteins

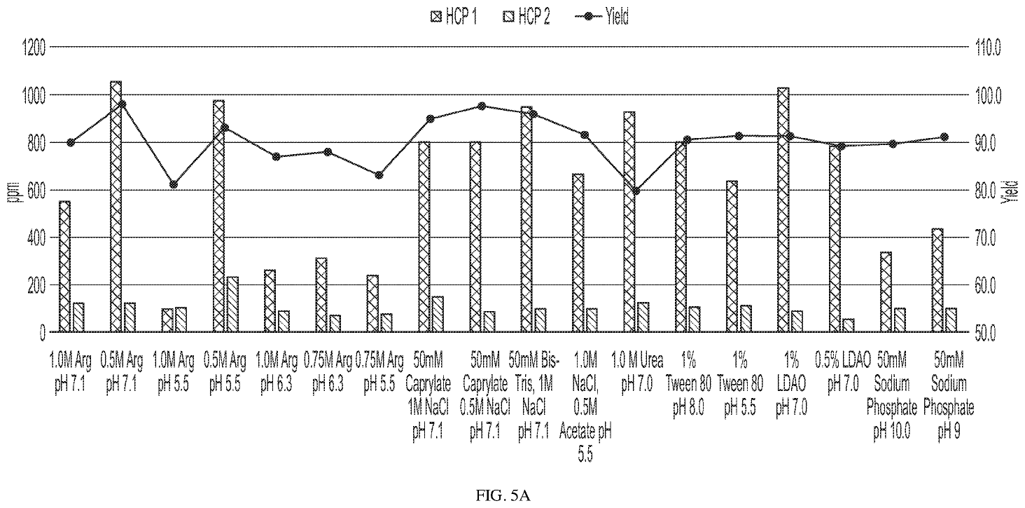

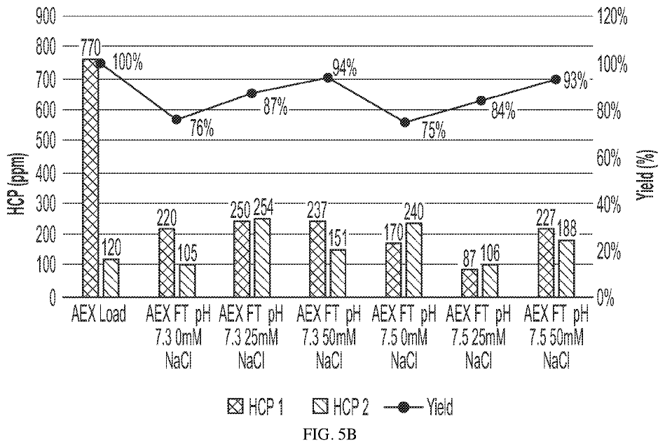

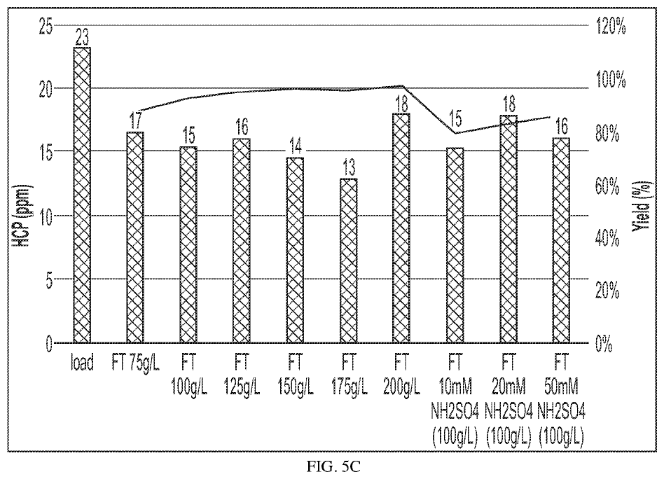

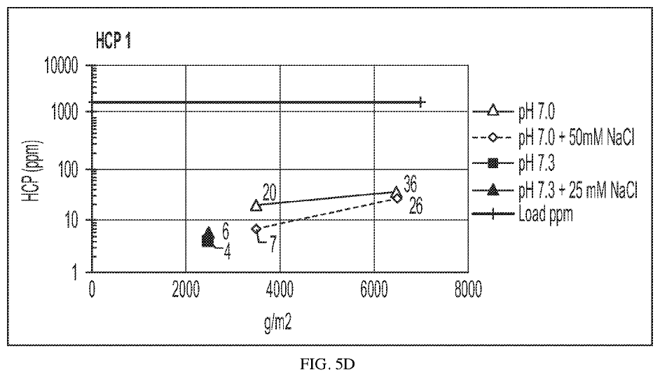

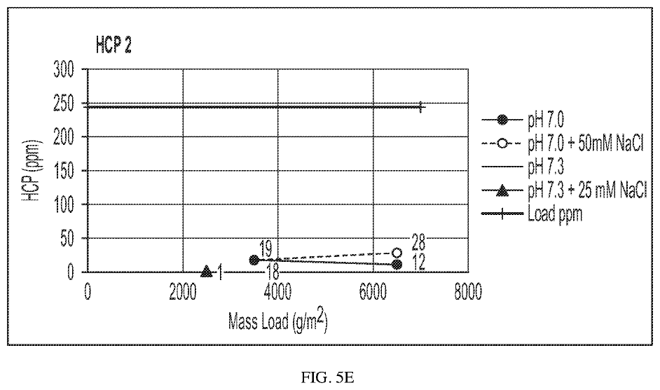

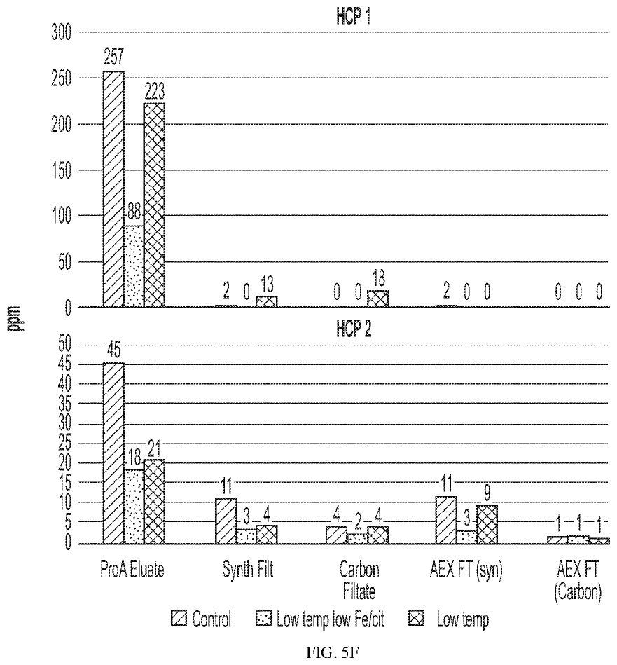

The upstream cell culture process described above reduced HSP90 and perilipin level, and downstream purification process can be optimized to remove the remaining HSP90 and perilipin. Various chromatography methods were tested for their capability of reducing HSP90 and perilipin. It was found that protein A column wash was effective in reducing perilipin under high arginine concentration at low pH with a yield loss, and was not effective in reducing HSP90 (FIG. 5A); anion exchange chromatography (AEX) was effective in removing perilipin but not HSP90 (FIG. 5B); and hydrophobic interaction chromatography (HIC) was not effective in removing HSP90 (FIG. 5C). Previously, synthetic depth filtration step was used between the protein A wash and the AEX chromatography. During the optimization process, carbon depth filtration was compared to synthetic depth filtration. To understand the conditions of how carbon depth filter can impact the removal of HCPs, different pH and salt concentrations were tested. It was found that loading mass has the largest impact on clearance of perilipin: perilipin was below the limit of quantification (LOQ) at loading mass of 3500 g/m² at pH 7.3 in 25 mM of NaCl (FIG. 5D). For HSP90, loading mass and salt have the largest impact on the clearance: HSP90 was below LOQ at loading mass of 2500 g/m2 in 25 mM of NaCl (FIG. 5E). An experiment was then designed to compare synthetic depth filtration to carbon depth filtration. In this experiment, the HCCF was first subjected to Protein A wash under high mass load. Subsequently, synthetic depth filtration or carbon depth filtration (4500 g/m²) at pH 7.3 in 25 mM NaCl was performed on the Protein A elute. The filtrates were then subjected to AEX chromatography (425 g/L) at pH 7.3 in 25 mM NaCl. The level of HSP90 and perilipin was tested after each run. The results showed that carbon and synthetic depth filters are effective in removing perilipin. The carbon depth filter cleared more HSP90 compared to the synthetic filter. (FIG. 5F).

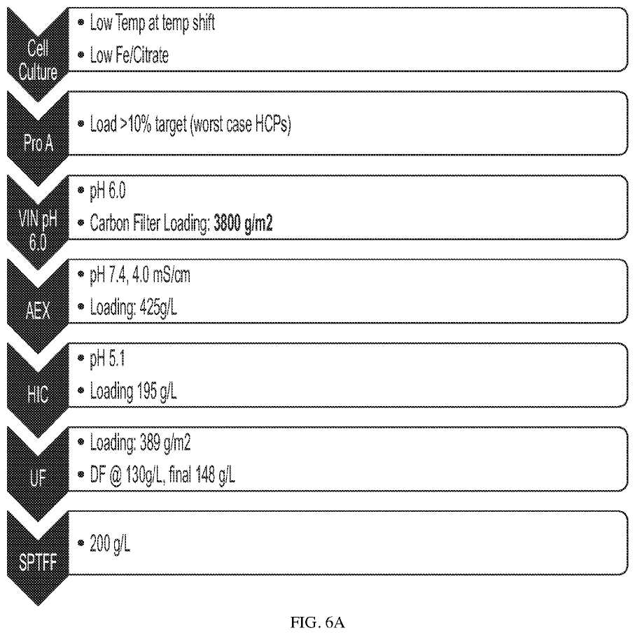

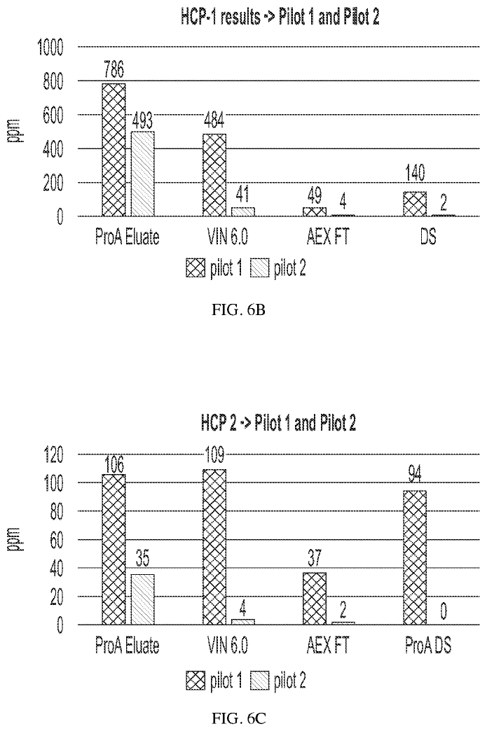

Accordingly, a pilot scale run was designed to combine the modification of the upstream cell culture condition and downstream purification process to remove HSP90 and perilipin from the drug substance. The flow of the run is described in FIG. 6A. Perilipin (FIG. 6B) and HSP90 (FIG. 6C) levels were tested after each step. FIGS. 6B-6C show the levels of HCP-1 (perilipin) and HCP-2 (HSP90) in control conditions run (pilot 1) and after the process was optimized for HCP reduction in “pilot 2”. During process optimization, the cell culture medium was modified by reducing levels of iron and citrate (ferric citrate), the culture temperature was reduced as described above in Example 2, and carbon depth filters were used in purification. As a result of these process levers used in combination, the amount of HSP90 and perilipin were reduced in the overall process (e.g., HCCF, all in-process intermediates, and the drug substance) (FIGS. 6B-6C).

### EQUIVALENTS AND TERMINOLOGY

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes instances in which exactly one member of the group is present in, employed in, or otherwise relevant to a given product or process. The disclosure includes instances in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or instances of the disclosure, is/are referred to as comprising particular elements and/or features, certain instances of the disclosure or instances of the disclosure consist, or consist essentially of, such elements and/or features. For purposes of simplicity, those instances have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that instances described in this document using an open-ended transitional phrase (e.g., “comprising”) are also contemplated, in alternative instances, as “consisting of” and “consisting essentially of” the feature described by the open-ended transitional phrase. For example, if the application describes “a composition comprising A and B,” the application also contemplates the alternative instances “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different instances of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such instances are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific instances described herein. The scope of the present instances described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other instances or portions thereof. The recitation of an embodiment herein includes that embodiment as any single embodiment or in combination with any other instances or portions thereof.

What is claimed is:

1. A method comprising:
culturing host cells comprising a gene encoding a recombinant protein in a cell culture medium, wherein the cell culture medium comprises:
(i) iron at a concentration of less than 1200 μM; and
(ii) citrate at a concentration of less than 2400 μM.

2. The method of claim 1, wherein the cell culture medium comprises iron at a concentration of between about 700 μM and about 1100 μM.

3. The method of claim 1 or claim 2, wherein the cell culture medium comprises iron at a concentration of between about 1000 μM and about 1100 μM.

4. The method of any one of claims 1-3, wherein the cell culture medium comprises citrate at a concentration of between about 1400 μM and about 2200 μM.

5. The method of any one of claims 1-4, wherein the cell culture medium comprises citrate at a concentration of between about 2100 μM and about 2200 μM.

6. A method comprising:
culturing host cells expressing a recombinant protein in a cell culture medium at a first temperature; and
decreasing the first temperature to a second temperature, wherein the second temperature is lower than 31° C.

7. The method of claim 6, wherein the host cells are cultured at the first temperature for at least 1 day prior to decreasing to the second temperature.

8. The method of claim 6 or claim 7, wherein the host cells are cultured at the first temperature for approximately 2 days prior to decreasing to the second temperature.

9. The method of any one of claims 6-8, wherein the decreasing of the first temperature occurs on day 3 of a production bioreactor process.

10. The method of any one of claims 6-9, wherein the first temperature is between about 34° C. and about 36° C.

11. The method of any one of claims 6-10, wherein the first temperature is 35° C.

12. The method of any one of claims 6-11, wherein the second temperature is between about 28° C. and about 30° C.

13. The method of any one of claims 6-12, wherein the second temperature is 29° C.

14. A method comprising:
(i) culturing host cells expressing a recombinant protein in a cell culture medium at a first temperature, wherein the cell culture medium comprises iron at a concentration of less than 1200 μM and citrate at a concentration of less than 2400 μM; and
(ii) decreasing the first temperature to a second temperature, wherein the second temperature is lower than 31° C.

15. The method of claim 14, wherein the cell culture medium comprises iron at a concentration of between about 700 μM and about 1100 μM.

16. The method of claim 14 or claim 15, wherein the cell culture medium comprises iron at a concentration of between about 1000 μM and about 1100 μM.

17. The method of any one of claims 14-16, wherein the cell culture medium comprises citrate at a concentration of between about 1400 μM and about 2200 μM.

18. The method of any one of claims 14-17, wherein the cell culture medium comprises citrate at a concentration of between about 2100 μM and about 2200 μM.

19. The method of any one of claims 14-18, wherein the host cells are cultured at the first temperature for at least 1 day prior to decreasing to the second temperature.

20. The method of any one of claims 14-19, wherein the host cells are cultured at the first temperature for approximately 2 days prior to decreasing to the second temperature.

21. The method of any one of claims 14-20, wherein the decreasing of the first temperature occurs on day 3 of a production bioreactor process.

22. The method of any one of claims 14-21, wherein the first temperature is between about 34° C. and about 36° C.

23. The method of any one of claims 14-22, wherein the first temperature is 35° C.

24. The method of any one of claims 14-23, wherein the second temperature is between about 28° C. and about 30° C.

25. The method of any one of claims 14-24, wherein the second temperature is 29° C.

26. The method of any one of claims 1-25, further comprising purifying the recombinant protein by a carbon depth filtration.

27. The method of any one of claims 1-26, wherein the host cells are mammalian cells.

28. The method of any one of claims 1-26, wherein the host cells are mammalian cells selected from the group consisting of: Chinese hamster ovary (CHO) cells, human embryonic kidney (HEK) cells, verda reno (VERO) cells, nonsecreting null (NSO) cells, human embryonic retinal (PER.C6) cells, Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells.

29. The method of any one of claims 1-28, wherein the recombinant protein is an antibody, an immunoadhesin, a Transforming Growth Factor (TGF) beta superfamily signaling molecule, a blood clotting factor, a Fc fusion protein, combinations thereof, or fragments thereof.

30. The method of any one of claims 1-29, wherein the method reduces host cell protein levels by at least 30%.

31. The method of claim 30, wherein the host cell protein is heat shock protein 90 (HSP90) and/or perilipin.

32. The method of any one of claims 1-31, wherein the cell culture medium is a chemically-defined cell culture medium.

33. The method of any one of claims 1-32, wherein the cell culture medium is a serum-free cell culture medium.
